(19) United States
(12) Patent Application Publication
(10) Pub. No.: US 2020/0022603 A1
(43) Pub. Date: Jan. 23, 2020

(54) SEMIOLOGY OF SEIZURES INCLUDING MUSCLE SIGNALS COLLECTED FROM ELECTROENCEPHALOGRAPHY ELECTRODES

(71) Applicant: Brain Sentinel, Inc., San Antonio, TX (US)

(72) Inventors: Damon P. Cardenas, San Antonio, TX (US); Jose E. Cavazos, San Antonio, TX (US); Michael R. Girouard, Shavano Park, TX (US); Jonathan J. Halford, Mount Pleasant, SC (US); Luke E. Whitmire, San Antonio, TX (US)

(21) Appl. No.: 16/465,841

(22) PCT Filed: Dec. 2, 2017
(86) PCT No.: PCT/US2017/064377

§ 371 Date: May 31, 2019

Related U.S. Application Data
(63) Continuation of application No. 15/491,883, filed on Apr. 19, 2017.
(60) Provisional application No. 62/429,359, filed on Dec. 2, 2016.

(51) Int. Cl. A61B 5/04; A61B 5/0488; A61B 5/00

(52) U.S. Cl. CPC A61B 5/04014; A61B 5/0488; A61B 5/7264; A61B 5/726; A61B 5/4094

(57) ABSTRACT

Methods and apparatuses for detecting and characterizing seizures are described. In some embodiments, the methods and apparatuses include collecting an EEG signal and selecting or filtering the signal in order to increase a prevalence of a part of said EEG signal derived from activation of muscle. In some embodiments, one or more EEG signals may be analyzed with one or more algorithms designed to detect muscle components of the signal in order to perform seizure semiology and/or to differentiate detected seizures based on type.

### BACKGROUND

A seizure may be characterized as abnormal or excessive synchronous activity in the brain. At the beginning of a seizure, neurons in the brain may begin to fire at a particular location. As the seizure progresses, this firing of neurons may spread across the brain, and in some cases, many areas of the brain may become engulfed in this activity. Seizure activity in the brain may cause the brain to send electrical signals through the peripheral nervous system activating different muscles of the body.

Seizures may characterize a number of distinct or related disease states. For example, seizures may be identified not only in patients with epilepsy, but also in patients who suffer from other disorders, including states characterized by psychogenic non-epileptic seizures (PNES). Notably, it may be particularly difficult to diagnose whether a patient is suffering from epilepsy, PNES symptoms, or both. Currently, EEG monitoring combined with video recordings (video-EEG) is considered the preferred way of identifying whether a patient may be experiencing epilepsy and/or PNES symptoms. However, even with video-EEG monitoring, it may sometimes be difficult to diagnose a patient with a specific condition. Accordingly, there remains a need for improved methods of diagnosing patients based on EEG data with or without corroborating video data. For example, there remains a need for improved methods of combining EEG data with other techniques to improve capability for identification of PNES.

EEG techniques generally focus on electrical activity associated with neuronal activation. Where electrical signals related to motor muscle activity are collected together with neuronal signals, such signals are generally considered as unwanted or noise signals. Although not typically done in clinical diagnosis, it may be advantageous to analyze a patient for signals associated with both activation of muscle fibers and neuronal activation. This may be done by collecting an electromyography (EMG) signal using electrodes placed on or near the skin, over a muscle, to detect electrical activity resulting from muscle fiber activation. To measure signals associated with muscle activation, sensor electrodes may be placed over one or more peripheral muscles, such as the biceps, triceps, or quadriceps. Accordingly, a distinct set of electrodes and associated collection system separate from EEG may be used. However, such systems are not typically used, and most clinical diagnoses of seizures involve video-EEG without any attempt to measure muscle activation during seizure events. There remains a need for improved methods of combining EEG with methods for measuring muscle activity, including methods that may not use an additional set of electrodes or other complicated instrumentation beyond which may be used for EEG signal detection.

### SUMMARY

In some embodiments, seizure detection systems and methods herein may use a portion of an EEG signal associated with muscle activity, which is typically considered a contaminant, in order to detect and/or characterize seizure events. In some embodiments, systems and methods may be used to collect and analyze component of an EEG signal for both activity associated with muscle activation and activity derived from the brain. In some embodiments, systems and methods may be used in an epilepsy monitoring unit (EMU) or other setting without the need for additional electrodes, or associated apparatuses, having to be placed on the patient for recording muscle activity.

In some embodiments, systems and methods herein may be used for measuring the duration of the tonic phase of a GTC seizure and/or other parts of a seizure. Notably, the durations of the phases of a GTC seizure may be recorded and quantified, and may be associated with various patient risk factors, including, for example, risk of sudden unexplained death from epilepsy (SUDEP). In some embodiments, other characteristics of GTC seizures or suspected GTC seizures, including, for example, overall intensity of the seizure, intensity of individual phases of the seizure, features of clonic-phase burst progression, other characteristics, and any combinations thereof may also be determined. In some embodiments, systems and methods herein may include analysis of seizure characteristics in order to establish whether a patient may have experienced one or more seizure events of a certain type. In some embodiments, systems and methods herein may include analysis of seizure characteristics in order to establish whether a patient should be further tested or evaluated in order to diagnose the patient as having a certain medical condition.

### BRIEF DESCRIPTION OF THE DRAWINGS

FIG. 1 is a flowchart illustrating some embodiments of a method for processing an EEG signal.

FIG. 2A shows muscle activity data for a patient.

FIG. 2B shows normalized muscle activity data for a patient

FIG. 3 is a schematic illustration of bins of data grouped together as may be used in a method of analysis of an EEG signal.

FIG. 4 shows a transfer function for an envelope filter.

FIG. 5 is a flowchart illustrating some embodiments of a method for classifying seizure events.

FIG. 6 is a flowchart illustrating some embodiments of another method for classifying seizure events.

FIG. 7 is a flowchart illustrating some embodiments of a method for collecting and processing an EEG signal.

FIG. 8 is a flowchart illustrating some embodiments of a method for characterizing seizure events.

FIG. 9 is a graph showing results for various durations widths of different parts of a seizure based on processing video data, EEG data, and EMG data.

FIG. 10 is a schematic diagram of a system for collecting or analyzing an EEG signal.

### DETAILED DESCRIPTION

The following terms as used herein should be understood to have the indicated meanings.

When an item is introduced by “a” or “an,” it should be understood to mean one or more of that item.

“Comprises” means includes but is not limited to.

“Comprising” means including but not limited to.

“Computer” means any programmable machine capable of executing machine-readable instructions. A computer may include but is not limited to a general-purpose computer, microprocessor, computer server, digital signal processor, or a combination thereof. A computer may comprise one or more processors, which may comprise part of a single machine or multiple machines.

The term “computer program” means a list of instructions that may be executed by a computer to cause the computer to operate in a desired manner.

The term “computer readable medium” means an article of manufacture having a capacity for storing one or more computer programs, one or more pieces of data, or a combination thereof. A computer readable medium may include but is not limited to a computer memory, hard disk, memory stick, magnetic tape, floppy disk, optical disk (such as a CD or DVD), zip drive, or combination thereof.

The term “designated EEG seizure data” as used herein means EEG signal previously identified by one or more caregivers as being associated with one or more seizures. For example, a caregiver may identify an EEG signal as associated with an actual seizure based on EEG data, video data, and or other data. A seizure event included among designated EEG seizure data may be referred to as a designated EEG seizure event.

The term “electroencephalography signal” or “EEG signal” as used herein refers to a signal produced from electrodes attached to the scalp or head of a patient, irrespective of the type of tissue from which the signal originated. An EEG signal may include or exclude one or more parts of a collected or stored signal. For example, an EEG signal may be a filtered signal that is limited to include one or more frequency components, or an EEG signal may be filtered or limited in some other way. In some embodiments, an EEG signal may refer to a signal derived or produced from data stored in permanent or transient computer memory or to the data itself. For example, where a collected EEG signal or part of a collected EEG signal is stored in a database, the stored data may be referred to as an EEG signal.

“Having” means including but not limited to.

The term “seizure-detection routine” refers to a method or part of a method that may be used to collect or analyze patient data and detect seizure activity or indicate increased risk that a seizure may occur or may have occurred. A seizure-detection routine may be run individually in a strategy for collecting patient data or may be run in combination with other seizure-detection routines or methods in an overall strategy for data characterization. One or more seizure-detection routines may sometimes be used to select or identify parts of EEG data that may include a seizure event. For example, one or more seizure-detection routines may sometimes be used to search for seizure or seizure-related activity in stored or archived EEG signal data.

The term “seizure event” as used herein, unless the context indicates otherwise, includes physiological events wherein a patient has suffered a seizure or exhibited physiological activity resembling the presence of a seizure.

The term “signal” as used herein refers to any form of energy that may transmit information and which is capable of being represented by data or as the data itself. Where a signal has been processed in order to provide the signal in a form that may be amenable for calculation or analysis, reference may sometimes be made to signal data.

Where a range of values is described, it should be understood that intervening values, unless the context clearly dictates otherwise, between the upper and lower limit of that range and any other stated or intervening value in other stated ranges, may be used within embodiments herein.

The apparatuses and methods described herein may be used to detect and characterize seizure or seizure-related events using one or more EEG electrodes or EEG sensors. As opposed to other techniques based on signals collected from EEG electrodes, methods herein may filter or select signals from EEG electrodes in order to focus on parts of the signal related to muscle activation. In some embodiments, the methods described herein may include collection and processing of one or more signals from one or more EEG sensor electrodes. In other embodiments, methods described herein may be used to characterize or analyze historical data, such as may be stored in one or more medical databases, by receiving a signal representative of the data. For example, methods described herein may exclude, or exclude use of, a sensor configured for signal collection, but may include, or include use of, one or more processors suitably configured to receive signal data and characterize seizure-related signals. Apparatuses described herein may, for example, include one or more EEG sensor electrodes suitable for collecting one or more EEG signals, one or more processors configured to analyze the signals, other apparatus components (e.g., components that may be used for storing or transmitting EEG signals), and combinations thereof.

In some embodiments, sensor electrodes may be positioned for measurement of electrical activity resulting from neuronal activity in the brain. For example, electrodes may be positioned on a patient's head or scalp. Electrodes configured or positioned for measurement of electrical activity of neurons in the brain are commonly referred to as EEG electrodes. As understood by those skilled in the art, the international 10-20 system may be used to describe the positioning of EEG electrodes over the head or scalp of a patient. Using the international 10-20 system, an EEG electrode may be described with a letter, which designates the lobe of the brain present underneath the positioned electrode, and a number, which describes a particular position of the electrode and hemisphere of the brain on which the electrode is positioned. In an EEG signal, signals related to muscle activity may also be present together with signals resulting from electrical activity derived from neuronal tissue in the brain. In typical EEG detection systems, such muscle-related signals are usually considered a contaminant or noise source obscuring the desired signal for analysis. Accordingly, processing suitable for reducing contaminant signals associated with muscle activity is commonly incorporated in many EEG methods. Surprisingly, in some embodiments herein, muscle-related-electrical activity may be detected using EEG electrodes or identified in EEG signal data and used to successfully characterize seizures or seizure-related events.

In some embodiments, one or more filters may be used to remove signals associated with neuronal activity from an EEG signal. In some embodiments, one or more filters may be used to focus on one or more parts of an EEG signal where muscle-related signal components may be present in high amounts or high relative amounts as compared to signal components associated with neuronal activity. In some embodiments, a resultant filtered EEG signal may be processed using additional steps in methods described herein. However, in some embodiments, EEG signal may be used in methods herein without specific filtering to remove electrical signals indicative of neuronal activity. In some embodiments, signal components associate with neuronal signals, as may be present in signal collected from one EEG electrode, may be estimated from signals collected by one or more other EEG electrodes. For example, an offset signal (e.g., related to brain activity) may be determined based on data from one or more EEG electrodes, and this offset signal may be used to estimate a signal correction applied to another EEG electrode.

In some embodiments, an EEG signal may be collected and/or analyzed using one or more EEG electrodes, wherein the position(s) of the one or more EEG electrodes include one or more of any of the standardized positions designated in the international 10-20 system. In some embodiments, EEG electrode data may be collected and/or analyzed using one or more EEG electrodes, wherein the position(s) of the one or more EEG electrodes is selected from one or more of the F7, F8, T3, and T4 positions as defined in the international 10-20 system. It may be noted that the aforementioned positions place the EEG electrodes in close proximity to the frontalis and temporalis muscles of the head. In some embodiments, EEG signals may be collected and/or analyzed using one or more EEG electrodes, wherein the position(s) of the one or more EEG electrodes is suitable to collect electrical data from brain neuronal tissue and one or more muscles of a patient's head or scalp. For example, in some embodiments, EEG electrodes may be placed directly over the frontalis and temporalis muscles or placed in some other way to enhance the strength of muscle-related signal components or relative strength of muscle-related signals versus other signal components.

In some embodiments, EEG signals may be processed and used to determine values of characteristics of seizure events which may be included in a quantitative summary of seizure activity. That information may then be provided to one or more caregivers. For example, a quantitative summary of characteristics of a detected seizure may be created and may include, by way of nonlimiting example, the duration of phases or parts of the seizure, including the tonic phase, clonic phase, entire seizure, and any combinations thereof. In some embodiments, the intensity or normalized intensity of one or more phases of a seizure or of an entire seizure may also be determined. In some embodiments, other characteristics associated with seizure events, including, for example, statistical metrics of qualified clonic-phase bursts, such as described in Applicant's copending U.S. application Ser. No. 14/920,665, may also be determined. For example, methods explicitly described herein may sometimes be executed together with routines described in various references incorporated herein, including, for example, routines which may be suitable to count qualified clonic-phase bursts and to provide a statistical summary of qualified clonic-phase bursts to one or more caregivers. In some embodiments, where events may be characterized and determined to be non-seizure events, a statistical summary of non-seizure events may also be provided to one or more caregivers. In some cases, a statistical summary of seizure characteristics may be prepared and organized for review by a medical professional. In some embodiments, a statistical summary may, for example, be used by medical professional to select whether a patient may be further tested in order to diagnose the patient as suffering from a medical condition, such as epilepsy or a condition wherein the patient may suffer from PNES symptoms.

Events that may be detected herein include generalized tonic-clonic seizure (GTC) events which may be caused by epilepsy. Detection of an event, unless the context indicates otherwise, should not be limited to detection in real-time. For example, where an event is identified by scanning historical or prior collected data, identification of the event as a seizure or as an event indicating increased probability of a seizure may be referred to as a detection.

Some of the embodiments described herein may be used to detect other types of seizures including some that may result from conditions other than epilepsy. For example, some seizures that may share one or more characteristics with generalized tonic-clonic (GTC) seizures commonly associated with epilepsy, such as increased muscle activity or increased repetitive muscle activity, may also be detected. For example, in some embodiments, PNES events may be detected and classified as events that may be indicative of a patient condition other than epilepsy. In some embodiments, complex-partial seizures may be detected and classified as resulting from a complex-partial seizure.

In some embodiments, methods described herein may analyze an EEG signal for frequency components associated with muscle activation and that may change during the course of a seizure. For example, methods herein may collect electrical signals derived from one or more detection sensor electrodes and process the signals to detect one or more high frequency components of an electrical signal, including signals above about 100 Hz. Methods herein may further process signals to detect one or more lower frequency components of an EEG signal, including signals less than about 75 Hz. In some embodiments, lower frequency components of an EEG signal may be monitored that may sometimes be indicative of muscle fatigue and/or of changes in a distribution of muscle fibers that may relate to transition between the tonic and clonic phases of a seizure. For example, in some embodiments, EEG signals may be collected and processed to identify frequency components between about 20 Hz to about 75 Hz. In some embodiments, low frequency components of EEG signal typically associated with repetitive motion, which may be identified during parts of the clonic phase of a seizure, may sometimes be excluded from one or more frequency bands used to indicate transition between the tonic and clonic phases of a seizure. For example, in some embodiments, frequency bands less than about 20 Hz or less than about 10 Hz may sometimes be excluded from one or more frequency bands. Accordingly, low frequency sources of noise, which may sometimes be difficult to fully remove or discriminate from muscle activity may be avoided or removed with high efficiency. High sensitivity detection or prediction of transition into the clonic phase of a seizure may then be made and/or made with minimal temporal lag between detection of phase transition and physical manifestation of the clonic phase.

In some embodiments, wavelet processing as described herein may be used to transform signal data to configure the data for detection of data features that may manifest over different frequencies and/or at different times. For example, by compressing or stretching various wavelets based on a basic or mother wavelet, wavelets may be configured that may be convolved with signal data to help identify signal features that may manifest at different frequencies and which may change over time. In some embodiments, one or more wavelet transforms may be used to transform EEG signal data to a form suitable for processing in order to detect the presence of one or more phases of a seizure or to detect a transition time between two phases of a seizure. For example, in some embodiments, a signal may be processed using a Morlet wavelet transform, Haar wavelet transform, Daubechies wavelet transform, harmonic wavelet transform, other suitable wavelet, or any combinations thereof.

Some wavelet transforms may provide for a more accurate reconstruction of input data than other transforms. However, generally, those wavelet transforms may demand somewhat greater processing resources than use of other wavelet transforms. Selection of one or more wavelet techniques may, in some embodiments, be based on those considerations and/or other considerations as described herein, including, for example, whether a method may be applied in real-time detection of seizures or in post-detection processing of stored or historical EEG signal data.

In a wavelet transform signal data may be represented by a group of functions based on one or more mother wavelets. Generally, a mother wavelet may be represented schematically as shown in Equation 1.

∫ψ(t)dt=0[Limits+∞/−∞] Equation 1

A group of functions may be generated from a mother wavelet by applying different scaling factors, which may be used to compress or stretch the mother wavelet. Other factors may be used to translate functions over time. For example, as shown schematically in Equation 2, a group or family of functions may be created from a mother wavelet using the factors a and b.

Ψ_{a,b}(t)=1/[a^{1/2}]ψ[(t−b)/a] Equation 2

By varying the factors a and b, a series of functions may be created as suitable to focus on different frequency components of a signal.

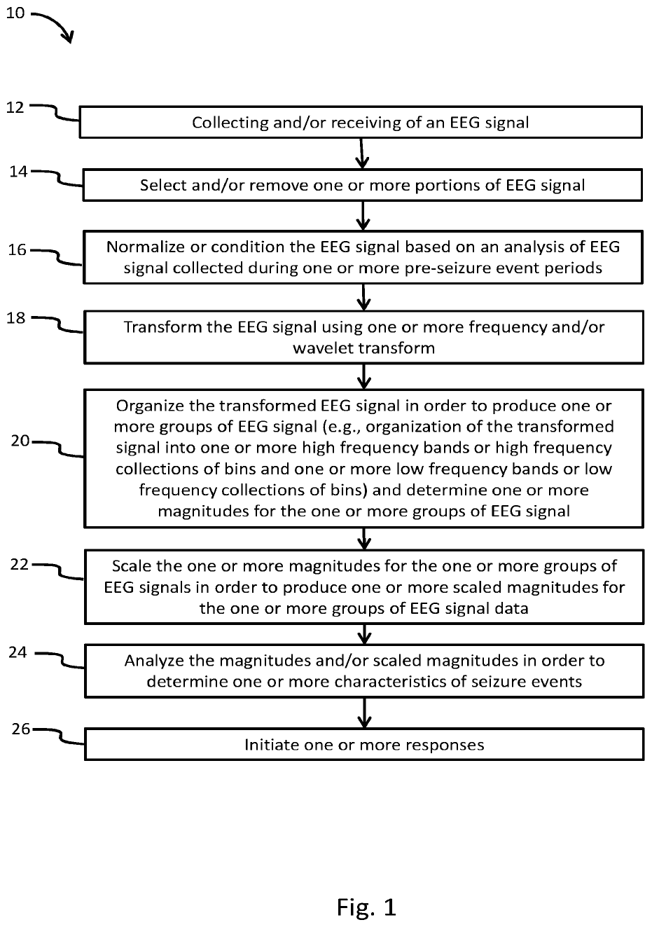

FIG. 1 illustrates some embodiments of a method 10 for collection and analysis of an EEG signal or for analysis of an EEG signal. Referring to step 12, in some embodiments, method 10 may include receiving an EEG signal for analysis. For example, method 10 may include receiving an EEG signal included in one or more databases including patient medical data. In some embodiments, EEG signal stored in a database may include portions of data identified or marked as being associated with one or more patient seizures. For example, an EEG signal may be designated EEG seizure data and may include one or more designated EEG seizure event. However, in some embodiments, methods herein may process or be capable of processing a raw or processed EEG signal for detection of seizure events that may not have been previously identified. For example, stored EEG signal may be processed using one or more seizure detection routines, and results derived from processing may be used to flag or select a part of the signal suitable for seizure characterization. In some embodiments, method 10 may include collecting an EEG signal from a patient. The collected EEG signal may then be processed to detect and/or determine characteristics of patient seizures. In some of those embodiments, collection and processing of signals may be executed in real-time and may, for example, be used to initiate an alarm or other response.

Collection of an EEG signal may include disposing one or more electrodes on a patient's head or scalp. For example, in some embodiments, one or more EEG electrodes may be positioned at one or more of the F7, F8, T3, and T4 positions as defined in the international 10-20 system. In some embodiments, EEG electrodes may be positioned in order to collect signal derived from one or more of the frontalis and temporalis muscles, or from some other muscle near the scalp or head of a patient. For example, EEG electrodes may be positioned near one or more muscles of a patient in order to optimize signal or signal-to-noise for detection of muscle-related components of an EEG signal. EEG electrodes may be suitably configured to transduce energy associated with physiological activity into a form that may be electronically processed. In embodiments where signals may be received for analysis (e.g., by accessing one or more medical databases), EEG signal received or selected for processing may be chosen in order to increase a prevalence of a part of said EEG signal that was derived from activation of muscle activity. For example, selected signal may be signal derived from one or more EEG electrodes positioned at one or more of the F7, F8, T3, and T4 positions.

In some embodiments, EEG signals may be further processed in order to provide the one or more signals in a form representative of levels of muscle activity. For example, in some embodiments, a signal may be amplified and processed using an analog-to-digital converter in order to produce signal data that may, for example, be used to express an amplitude, magnitude, or power content of muscle activity. In some embodiments, a signal may be processed in one or more operations in order to shape, condition, or isolate one or more frequency bands associated with a part of an EEG signal in order to provide muscle activity data associated with one or more frequency bands or regions. For example, an EEG signal may include frequencies over some frequency range, and a part of the EEG signal may include signal components in one or more particular frequency ranges or bands. In some embodiments, an EEG signal may be rectified, filtered, or other operations may be used to shape, condition, or isolate a desired part of an EEG signal. For example, one or more frequency band where muscle activity may be present in high amounts or in high amounts relative to signals derived from brain tissue may be isolated.

In some embodiments, isolation of a part of an EEG signal in one or more frequency bands may, for example, include use of one or more filters. Filtering may, for example, be achieved using software or electronic circuit components, such as bandpass filters (e.g., Baxter-King filters), suitably weighted. However, such description should not be interpreted as limiting methods herein to filtering with either software or electronic circuit components. For example, in some embodiments, analog or digital signal processing or combinations of analog and digital signal processing may be used for isolation of frequency band data.

In some embodiments, as shown in step 14, one or more portions of an EEG signal may be selected and/or removed from other parts of the EEG signal. For example, portions of an EEG signal associated with one or more collection times may be separated from EEG signal collected at other times. Thus, in some embodiments, processes in steps 12, 14 may both isolate one or more parts of a signal related to components of the signal derived from muscle activity (step 12), and also isolate portions of the signal collected during certain times (step 14). In some embodiments, selection of one or more portions of signal in the step 14 may include detecting, at least at some level of probability, one or more seizure events.

Selection of EEG signal in step 14 may include use of one or more algorithms designed to analyze the signal for seizure activity. Suitable algorithms may be based on methods of seizure detection using conventional EEG (e.g., any of a number of available EEG algorithms for detecting seizures based on detected brain activity may be used), algorithms designed for detection of muscle activity, other available methods, or any combinations thereof. Once a seizure event or suitable number of seizure events is detected, one or more parts of EEG signal may be selected. For example, one or more parts of EEG signal may be selected, wherein the parts may include or may be temporally associated with one or more detected seizure events. For example, signal preceding a detected seizure event, including a detected seizure event, following a detected seizure event, or combinations thereof may be selected. In some embodiments, selected signal may precede or immediately precede a time or time range when a seizure event was detected. Other factors, including, for example, amplitude levels of signal data or the absence of transient spikes of signal data, may also be used in selection of data.

In some embodiments, detection of a seizure event may indicate a high confidence that a true seizure (e.g., a generalized tonic-clonic seizure or other seizure type commonly associated with epilepsy and/or the seizure disorder) may be occurring or may have occurred. And, in some embodiments, processing of signal using both one or more processing algorithms designed for detection of brain activity and one or more algorithms designed for analysis of muscle activity may be run together, such as to increase confidence of seizure event detection. However, in some embodiments described herein, selection of signal data in step 14 may include detection of one or more seizure events which, while showing signs of being associated with a seizure, may or may not indicate the presence of an actual seizure or that a seizure was detected at high confidence. For example, in some embodiments, selection of data in step 14 may include detection of one or more signal amplitudes or detection of a rate of change of signal amplitude that may be elevated above some suitable threshold level. Those changes may indicate an increased risk of seizure occurrence, but the changes may be insufficient to fully discriminate signals from some non-seizures sources which may also produce elevated signal data. Further processing in additional steps beyond step 16 may then be used to characterize and increase confidence that selected data should properly be associated with an actual seizure. Alternatively, further processing in additional steps beyond step 16 may sometimes be used to identify that a detected event may properly be categorized as a non-seizure event. Thus, in some embodiments, processing of a collected signal may reduce the probability of false positive seizure detection. In some embodiments, further processing of signal in one or more additional steps that may be executed in addition to step 14 may be used to characterize a seizure already detected at high confidence. For example, seizure semiology and other characteristics may of a seizure may be determined in order to help classify a seizure as being either a GTC seizure, PNES seizure, or other type of event.

In some embodiments, one or more selected portions of EEG signal may further be removed or separated from a larger set of EEG signals. For example, one or more selected portions of EEG signal may be removed from a larger set of EEG signal collected during patient monitoring. Alternatively, one or more selected portions of EEG signal may be removed from a larger set of EEG signal included in a storage or other database. For example, in some embodiments, selection of EEG signal data in step 14 may include detecting an event indicating the presence or increased risk of a seizure using one or more processors included in a detection device disposed on or near one or more of a patient's muscles. Selected EEG signal associated with the event may then be isolated or removed from other signal for further processing and/or characterization. For example, selected EEG signal may be isolated from other EEG signals and sent to a remote processor such as may be included in a stationary base station where the selected signal may be further processed and characterized in greater detail.

In some embodiments, identification that a seizure may have occurred may serve to initiate selection of data in step 14 and act as a trigger or gate for further processing. For example, further processing (e.g., processing in additional steps of method 10) of selected EEG signal data may be useful to prevent resource allocation, including human and/or computational resource allocation, being unnecessarily spent on event characterization. In addition, in some embodiments, non-selective analysis and characterization of all signals, the majority of which may generally be non-seizure signals, may sometimes produce unreliable results. For example, indiscriminate processing and characterization of all EEG signal data may produce some spurious results that might have been avoided or removed if only selected EEG signal data was fully processed and/or characterized. Accordingly, selection of data in the step 16 may act as a screen wherein only significant EEG signal may be characterized.

Any of a number of suitable seizure-detection routines or combinations of seizure-detection routines may be used in selection of data in step 14. For example, in some embodiments, any seizure-detection routine that may be used to detect a time or time range for the start of seizure activity, at least to some level of temporal resolution, may be applied in selection of signal data. Once a start time or time range for the start of a seizure is determined, a portion of data that includes the start of the seizure and/or a portion of data that includes one or more pre-seizure time periods may be selected or selected and removed from other signal.

For example, in some embodiments, if an event indicating a seizure or increased risk of a seizure is identified within an EEG signal, then an approximately 10-minute portion of data or data from some other suitable time period may be selected and removed from other EEG signal in step 14. A start time or time range for the event may be approximately centered or positioned in some other desired way within the selected portion of EEG signal data. Thus, pre-event periods of data (which may be referred to as pre-seizure periods) may be identified within about the first half of the selected data. One or more pre-seizure periods (e.g., time periods from about the first half of selected signal data in the above example) may then be identified within a selected portion of data and used in further processing. For example, statistical information calculated from the one or more pre-seizure periods may be used to normalize or condition EEG signal, as described below in the step 16.

In some embodiments, including some embodiments suitable for real-time seizure detection and seizure characterization, statistical information associated with EEG signal data may be continuously determined, or determined at certain intervals. And, for example, if an event indicating a seizure or increased risk of seizure occurrence is detected, one or more of the time periods (e.g., time periods associated with data for which statistical information was already calculated) may then be temporally oriented with respect to the detected event. For example, the one or more time periods may be identified as preceding a detected event, and may be designated as one or more pre-seizure periods which may be selected in step 14. Therefore, calculation of statistical information useful for normalizing or conditioning data in step 16 may sometimes be executed before pre-seizure time periods are selected in step 14. Accordingly, in some embodiments, some operations described in reference to step 16 (e.g., calculation of pre-seizure statistical information) may be executed or executed in-part together with or before selection of data in the step 14.

In some embodiments, an about 10 second period to an about 200 second period of data may be included in a pre-seizure time period. One or more pre-seizure periods of signal data may be located within an overall pre-seizure portion of data and may be temporally located with respect to a time or time region associated with when a seizure may have occurred. In some embodiments, pre-seizure time periods may be selected that may be immediately or closely followed by a seizure. For example, in some embodiments, one or more pre-seizure time period may be selected including time periods that may be within about 10 minutes, within about 5 minutes, or within about 1 minute from the start of a seizure.

Detection of an event indicating a seizure or increased risk of a seizure, as may be used to select data in the step 14 or for other purposes herein, may include use of any appropriate seizure-detection routine or combination of seizure-detection routines. For example, in some embodiments, a seizure-detection routine may analyze signal data collected over a time period and examine the signal to look for one or more amplitude values that may exceed a threshold amplitude. In some embodiments, a seizure-detection routine may include examining EEG signal collected over some time period and determining whether one or more data values derived from the EEG signal exceed one or more thresholds within one or more time windows within that period. And, based, for example, on a number of time windows or consecutive time windows in which the one or more thresholds were exceeded, a seizure may be detected. In some embodiments, EEG signal may be integrated, and one or more integrated values may be compared to a threshold.

In some embodiments, EEG signal may be processed in other ways in order to facilitate detection of a seizure event or event indicating increased risk of a seizure. For example, in some embodiments, the magnitude of a statistical value related to levels of muscle activity and processed from EEG signal isolated in one or more frequency bands may be determined. For example, in some embodiments, a statistical value may be a T-squared statistical value that may not only be related to levels of muscle activity but may also be more sensitive to seizure activity than other values related to muscle activity, including, for example, power content determined from one or more bands. Methods of calculating T-squared statistical values from muscle activity data, within the context of EMG, are described in detail in Applicant's U.S. Pat. No. 9,186,105 and U.S. Pat. No. 9,439,596 each of which are incorporated herein by reference. Methods therein, described in the context of EMG, may be applied in some embodiments herein where muscle activity data is detected in an EEG signal.

In some embodiments, one or more seizure detection routines used in the step 14 may be configured to provide early detection of seizure activity. For example, some of those routines may be configured to identify seizure activity without substantial delay and may be configured to send an immediate warning to one or more caregivers if seizure activity is detected. In some of those embodiments, methods herein may sometimes be configured to update an emergency response as more information is gained about a seizure event. For example, in some embodiments, if an event characterized in the method 10 is found to be a non-seizure event or seizure event related to a condition other than epilepsy an emergency response may sometimes be adjusted, such as in part of a response initiated in step 26 described below. For example, a caregiver may be informed that further analysis of a detected event indicated that the event was not a seizure event. In some embodiments, including, for example, embodiments wherein a method 10 may be used to provide information that may be used to update an emergency response, selected data may include times about 1 minute, about 2 minutes, or about 5 minutes after a time or time range for which a seizure or possible seizure was identified. Thus, for example, at any of those intervals, method 10 may sometimes be used to cancel an alarm response (as described further in step 26).

In some embodiments, as described in step 16, one or more selected portions of EEG signal may be normalized or conditioned based on one or more EEG signal values calculated from EEG signal collected during one or more pre-seizure event time periods. In some embodiments, normalizing or conditioning of the one or more selected portions of EEG signal may include calculating an average or mean value of the amplitude of EEG signal in one or more pre-seizure event time periods and subtracting the calculated value from EEG signal collected during a seizure event. Alternatively, an appropriate statistical value related to a mean value, such as a median or mode value, may also be used. Thus, in some embodiments, EEG signal data for a seizure-related event may have any direct current (DC) offset signals substantially removed, thereby providing DC offset or corrected EEG signal data.

In some embodiments, normalizing or conditioning of the EEG signal may include dividing DC offset or corrected EEG signal (e.g., signal following the aforementioned subtraction) by a standard deviation or average standard deviation of EEG signal collected in one or more pre-seizure periods. In some embodiments, an appropriate statistical metric associated with the spread of a dataset (e.g., spread, variance, average deviation, or other suitable statistical metric) may be substituted for a standard deviation.

In some embodiments, a plurality of pre-seizure event time periods may be selected in step 14, and one or more statistical values may be calculated from EEG signal in each of the plurality of seizure event time periods in step 16. Where one or more statistical values of EEG signal data may be calculated in more than one pre-seizure event time periods, pooled statistical metrics (e.g., a pooled mean or other pooled value) may be determined. In some embodiments, trends in statistical values over time may also be determined. For example, in some cases the magnitude of a mean value of the amplitude of EEG signal data and associated level of DC offset suitable for a patient may change near the start of a seizure. And, for example, an extrapolated statistical value may be used to estimate one or more DC offset corrections or other values used to normalize or condition data.

In some embodiments, normalizing and/or conditioning of EEG signal data may facilitate improved comparison of EEG signal data between patients and/or between monitoring sessions. For example, it may be useful to normalize EEG signal data by removing DC offset signals and/or adjusting EEG signal data based on pre-seizure noise levels so that data between patients or between monitoring sessions for a single patient may be more accurately characterized in automated and/or semi-automated analysis embodiments of method 10. In some embodiments, one or more measurements of the spread or noise in a pre-seizure-related event time period may serve as a first order estimate for properties of the skin/electrode interface as configured during times when a seizure-related event was detected. And, the aforementioned measurements may serve as a first order correction for differences in collection efficiency of electrical signals between different patients and/or between different monitoring sessions for one or more patients. Thus, some metrics characterized herein, including, for example, metrics related to seizure intensity, may be more accurately determined.

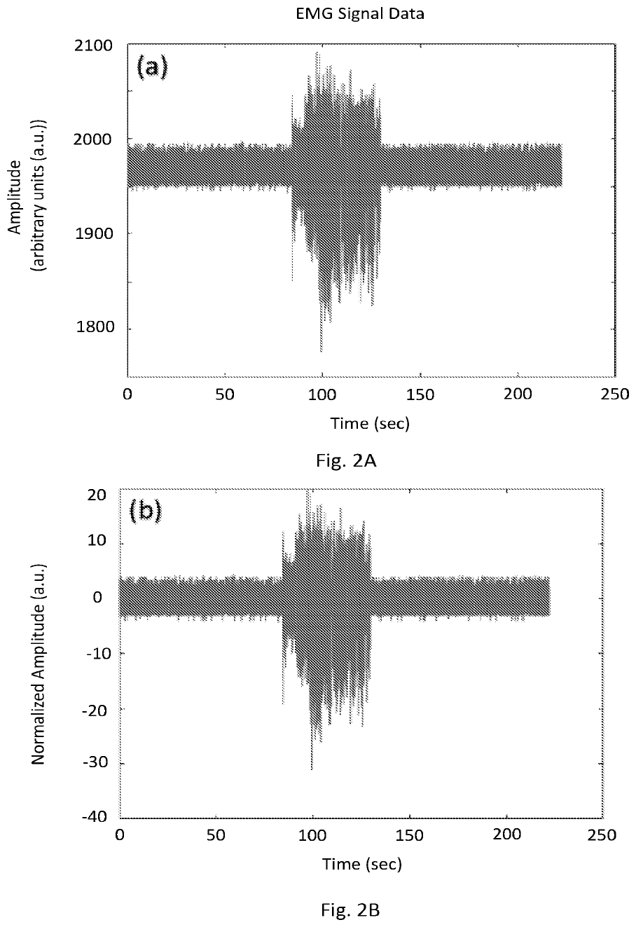

By way of example, FIG. 2A shows muscle activity data (collected with EMG electrodes) for a patient having a generalized tonic-clonic seizure as is apparent between t=75 seconds and t=140 seconds. FIG. 2B shows normalized muscle activity data following subtraction of a mean value calculated from a 100-second time period of pre seizure event data (not shown) from the data in FIG. 2A (i.e., wherein the signal data in FIG. 2A is the minuend and the mean value calculated from the 100-second pre-seizure data is the subtrahend) and division of the resulting difference by a standard deviation calculated from the same pre seizure event time period.

In step 18, EEG signal may be processed using one or more frequency and/or wavelet transforms. Where EEG signal or EEG signal data is referred to in step 18 (and additional steps executed following step 18 in the method 10), unless the context clearly indicates otherwise, EEG signal may refer to EEG signal which may or may not be normalized or conditioned as described above in step 16. Where embodiments are specifically limited to normalized or conditioned EEG signal data, the term “conditioned EEG signal data” will be used. In some embodiments, EEG signal data may be processed with a Morlet wavelet, which may be used to express the complex power in frequency over time of EEG signal data. In such an approach, a Morlet wavelet may be used to transform EEG signal data into a form suitable for calculating magnitudes at which frequency components of the signal may be present at about a given time (e.g., within a certain temporal resolution) or over a given time interval. The Morlet wavelet transform may be characterized by Equation 3 and Equation 4. That is, the wavelet transform used may be expressed as:

$$C ( a , b ; f ( t ) , ψ ( t ) = {\int }_{- ∞}^{∞} f ( t ) \frac{1}{\sqrt{a}} ψ ( \frac{t - b}{a} ) dt \qquad Equation 3$$

where a is a scaling factor and b is a shifting factor (see also Equation 2). In Equation 3, f(t) is the signal analyzed and ψ(t) is the wave function. For example, in some embodiments, f(t) may include EEG signal data normalized or conditioned as described in step 16. In some embodiments, f(t) may include one or more portions of selected EEG signal data from step 14. Thus, in some embodiments, normalized or conditioned EEG signal data may be processed in step 18 or selected data in step 14 may be processed. In some embodiments, the wave function used may be:

$$ψ ( s ω ) = {π}^{\frac{1}{4}} {e}^{- {( s ω - {ω}_{0} )}^{2}} / 2 U ( s ω ) \qquad Equation 4$$

In Equation 4, ω0 is the center frequency, sω is a scaled frequency and U(sω) is the Heaviside step function.

Application of a wavelet transform on a signal may, for example, be used to produce a three-dimensional dataset wherein time and frequency components of the signal may sometimes be represented along the x and y axes, respectively, and an estimate of the signal magnitude may be indicated in a third dimension, such as may be indicated in a color coded or contoured plot.

In some embodiments, as shown in step 20, transformed EEG signal may be organized in order to produce one or more groups of EEG signal. Organization of data may, for example, including filtering the data in order to select groups of data in one or more frequency band. Furthermore, in additional parts of step 20, one or more magnitudes of signals in the one or more groups of EEG signal may be determined. For example, grouping of signal data may produce one or more groups of EEG signal extending over one or more frequency ranges. And, the magnitude or amplitude of EEG signal in the one or more frequency ranges may then be determined.

In some embodiments, organization of transformed EEG signal and determination of one or more magnitudes of a group of EEG signal may, for example, include integrating the transformed EEG signal over one or more integration boundaries, or binning the transformed EEG signal and generating a sum of EEG signal data included in one or more created bins or collections of bins, or both.

A bin of EEG signal may refer to a segment of EEG signal bounded by a range of frequency and time. In some embodiments, integration or bin boundaries of transformed EEG signal for frequency, time, or both may be scaled against a resolution limit of the transformed EEG signal data. For example, one or more of the aforementioned boundaries may be scaled in about a proportion to a frequency resolution limit, a temporal resolution limit, or both. In some embodiments, integration or bin boundaries may include a first boundary for one variable (e.g., time or frequency) that is held constant, and the other (or second) boundary (e.g., other of time or frequency) may be scaled against the resolution of the transformed EEG signal data for measurement of that variable.

In some embodiments, organizing data in step 20 may include creation of a plurality of bins. For example, a plurality of bins may extend across all or some subset of a range of collected frequencies in an EEG signal. In some embodiments, a plurality of bins may be created wherein the plurality of bins may span one or more frequency ranges. For example, in some embodiments, about 190 bins (e.g., 193 bins) may be created. The bins may, for example, extend over a frequency range from about 3 Hz to about 420 Hz, or the bins may extend over some other frequency range described herein.

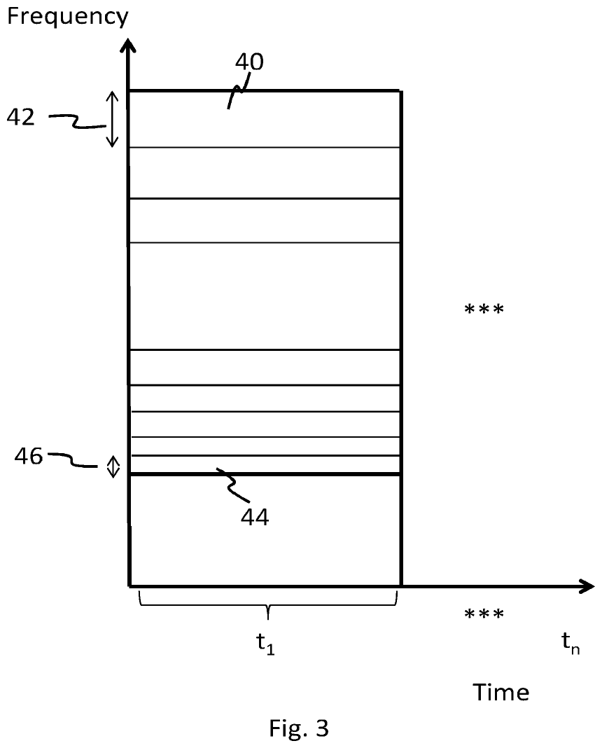

As described above, in some embodiments, bins herein may include a frequency and/or time range boundary that is varied across either or both of frequency and/or time. For example, bin boundaries may be varied across either or both of a frequency and/or time range in proportion to how the resolution in frequency and/or time of the wavelet transformed signal data may change. For example, for any fixed interval of time, processing of EEG signal with a wavelet transform may produce data with a frequency resolution that is greater in a low frequency range (where frequency resolution of the transformed signal may be higher) than in a high frequency range (where frequency resolution of the transformed signal may be lower). For example, as may be understood in reference to FIG. 3, a high frequency bin 40 may be configured to include data associated with the time interval (t1) and also associated with frequencies within a frequency range or interval 42. As also shown in FIG. 3, a lower frequency bin 44 may be configured to include data associated with the same time interval (t1) and also associated with frequencies within a frequency range or interval 46. The frequency range 46 associated with the bin 44 may include a narrower frequency range than the frequency range 42 associated with the bin 40. For example, the frequency ranges 42, 46 may be about proportional to a resolution limit for a signal included in each respective bin 40, 44. And, because the resolution limit in frequency is better at low frequencies, the range 46 is narrower than the range 42.

Still referring to step 20, in some embodiments, two or more collections of bins may be created from the transformed EEG signal. For example, in some embodiments, a first collection of bins (or high frequency collection) may include one or more bins included in a frequency range from about 150 Hz to about 260 Hz. A group of EEG signal data including a high frequency collection of bins may be referred to as a high frequency group of EEG signal data. In some embodiments, a high frequency collection of bins may include one or more bins that may include a lower frequency boundary of about 120 Hz, about 150 Hz, or about 180 Hz. In some embodiments, the high frequency collection of bins may include one or more bins that may include an upper frequency boundary of about 200 Hz, about 260 Hz, about 300 Hz, or about 400 Hz. In some embodiments, all collected high frequency signals, which may be relatively weak above 400 Hz for most patients, may be included in a high frequency set of bins. In some embodiments, a high frequency group of bins may be specifically used to enhance a prevalence of a part of said EEG signal derived from activation of muscle activity

A second collection of bins (or low frequency collection) may include one or more bins included in a range of frequencies from about 6 Hz to about 70 Hz. In some embodiments, a low frequency collection of bins may include one or more bins that may include an upper frequency boundary of about 60 Hz, about 50 Hz, or about 45 Hz. In some embodiments, a low frequency collection of bins may include one or more bins that may include a lower frequency boundary of about 10 Hz, about 20 Hz, or about 30 Hz. A group of EEG signal including a low frequency collection of bins may be referred to as a low frequency group of EEG signal. In some embodiments, a low frequency group of bins may be specifically used to enhance a prevalence of a part of said EEG signal derived from activation of muscle activity. In some embodiments including each of a high frequency and low frequency collection of bins, tonic and clonic phase seizure activity may be detected throughout the entire course of a generalized-tonic-clonic seizure. For example, two groups of EEG signal may be used or used exclusively to perform seizure semiology. In some embodiments, groups organized in step 20 may include a first group of EEG signal data including or made from a collection of one or more bins extending across a high frequency band. And, a second group of EEG signal data may include or be made from a collection of one or more bins extending across a low frequency band.

In some embodiments, a first group of EEG signal may include a high frequency collection of bins including one or more bins included in a range of frequencies above about 120 Hz. The high frequency collection of bins may sometimes include a high-frequency upper cut-off of about 400 Hz. More than one low frequency collection of bins may also be organized. For example, a first low frequency collection of bins may include one or more bins included in a range of frequencies from about 6 Hz to about 70 Hz. The lower frequency boundary of that first low frequency collection of bins may be about 10 Hz, about 20 Hz, about 40 Hz, or about 50 Hz. In some embodiments, one or more additional lower frequency collections of bins may be organized. For example, an additional low frequency collection of bins may include one or more bins included in a range of frequencies from about 2 Hz to about 10 Hz.

In some embodiments, in the step 20, one or more magnitudes of a high frequency group of EEG signal and one or more magnitudes of a low frequency group of EEG signal may be determined across one or more analysis time windows. In some embodiments, an analysis time window may, for example, extend across a time duration suitable to encompass pre-seizure time periods and the full duration of a typical GTC seizure or an expected duration for some other type of seizure event. In some embodiments, an analysis time window may extend across the full duration of EEG signal selected in the step 14.

In some embodiments, one or more magnitudes of EEG signal may be determined from one or more collections of bins. For example, in some embodiments, magnitudes of signal for at least one high frequency collection of bins and at least one low frequency collection of bins may be determined and tracked throughout all or some part of an analysis time window. For example, one or more bins may extend over a certain time increment or time unit in an analysis time window and also extend over one or more frequency ranges. In step 20, bin magnitudes may be summed across each bin in a collection of bins. This process may be repeated across time (e.g., for other time increments or time units within an analysis time window) to derive magnitude data for one or more collections of bins across time.

In some embodiments, one or more magnitudes of groups of EEG signal may be determined by integrating the transformed EEG signal data over boundaries with respect to frequency and/or time. For example, the transformed EEG signal data may be integrated over some increment or unit of time (e.g., an increment or unit of time within an overall analysis window) and over any of the aforementioned frequency ranges associated with any of various collections of bins as described above. The aforementioned integrations may be repeated for other time increments or time units within an overall analysis time window. Thus, an integrated magnitude or strength of signal in one or more bands may be tracked over any part of an analysis time window.

In some embodiments, a first group of EEG signal may, for example, include data from a collection of one or more bins extending across a high frequency band, such as a band ranging from about 150 Hz to about 260 Hz. A high frequency collection of bins may further include data extending across some increment of time. For example, a collection of bins may extend over the aforementioned frequency range and some increment of time, such as an increment of time from about 10 milliseconds to about 100 milliseconds. For any given increment of time, a collection of bins may be analyzed. For example, a suitable metric related to the magnitude of signals in the collection of bins may be determined. For example, the magnitude of signals may be determined using one or more of a sum, mean, or median value for the bins in a collection. This analysis may be repeated for other increments over an analysis time window. Similarly, magnitudes for one or more other groups of EEG signal, including, for example, groups extending across a low frequency band, may also be determined. That is, magnitudes of signals for bins extending over some frequency range and over an increment of time may be determined. The procedure may be continued for other increments extending across an analysis time window in order to produce EEG signal data across time.

In some embodiments, additional processing may also be performed in step 20. For example, in some embodiments, EEG signal may be smoothed, one or more DC offset or baseline corrections may be applied, or both. For example, one or more envelope filters may be applied in order to smooth magnitude data for one or more groups of EEG signal data. As referred to herein, magnitude data for one or more groups of EEG signal may refer to either magnitude data for smoothed EEG signal or magnitude data for EEG signal that has not been smoothed.

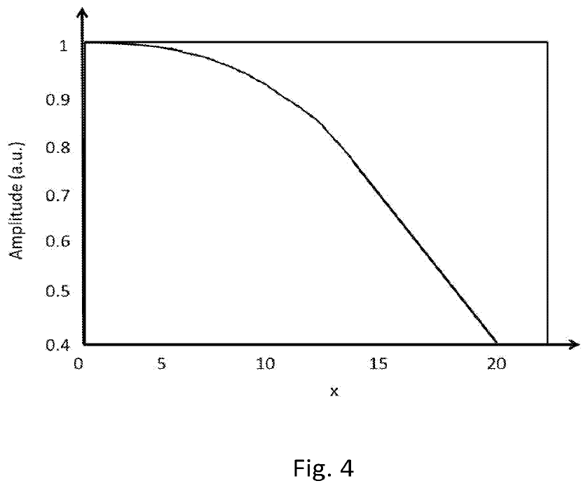

As described above, in some embodiments, EEG signal data for one or more groups of EEG signal data may be processed using one or more envelope filters. For example, a representative envelope filter suitable for use in some embodiments herein is described by the exponential decay function shown in Equation 5 and further shown in FIG. 4.

f(x)=e^{-0.002x}^{²}, where 0<x<20 Equation 5

In some embodiments, as shown in step 22, one or more magnitudes of one or more groups of EEG signal may be scaled in order to produce one or more scaled magnitudes for the one or more groups of EEG signal. For example, scaling of magnitude data may include dividing magnitude data for a group of EEG signal by a maximum magnitude value achieved for the group of EEG signal data over an interval of time, such as a time interval within an analysis time window or within a part of an analysis window.

For example, a maximum magnitude or strength may be calculated for each of a high frequency and a low frequency part of a signal. A maximum magnitude value may be an absolute maximum magnitude or a local maximum magnitude value. For example, in some methods where EEG signal data is evaluated in post-processing, collected EEG signal for a duration or full duration of a detected seizure-related event may be available to a processor when determining scaled magnitude data. Accordingly, an absolute maximum magnitude value may be readily assigned. However, in some embodiments suitable for real-time analysis, one or more local maximum magnitude values may be assigned and/or used to calculate scaled magnitude data. In some embodiments, methods herein may determine if a local maximum magnitude value meets requirements to be designated an absolute maximum magnitude value for EEG signal collected during a seizure-related event. For example, if a local maximum magnitude is maintained for greater than about 5 seconds to about 10 seconds (i.e., no other adjacent or following value exceeds the local maximum magnitude value), then the local maximum magnitude may be designated as an absolute maximum magnitude value. In some embodiments, other information, such as the slope or shape of EEG signal data on either side of a local/absolute maximum magnitude value may also be used in determining if the magnitude value is designated as a local or absolute magnitude value.

Further by way of example, in order to scale magnitude data (step 22 of FIG. 1), a magnitude of data for the high frequency collection of bins may be scaled by dividing the data by the maximum magnitude achieved within a high frequency dataset. Likewise, the magnitude of data for a low frequency collection of bins may be divided by a maximum magnitude achieved within the low frequency dataset.

As shown in step 24 of FIG. 1, an analysis of magnitudes and/or scaled magnitudes may be executed in order to characterize seizure events. In some embodiments, characteristics of seizure events that may be determined in step 24 may include, by way of nonlimiting example, the duration of phases or parts of seizure events, event type, event intensity, and combinations thereof. In step 26, one or more responses may be initiated based, for example, on the identified characteristics of a seizure-related event. In some embodiments, step 24 and/or step 26 may include execution of one or more of the methods 90 and 110. For example, one or more of those methods (or one or more steps in those methods) may be executed as one or more sub-routines of method 10.

In some embodiments, analysis in step 24 may include comparison of one or more magnitudes or scaled magnitudes of one or more groups of EEG signal to one or more thresholds. And, based on the comparison of magnitude and/or scaled magnitude signals to one or more thresholds, one or more phases of seizure activity may be determined. For example, some of the embodiments herein may include detection of the presence of clonic-phase activity, tonic-phase activity, or both. Classification of seizure-related events may then include an evaluation of whether one or more of the aforementioned phases were detected. In some embodiments, transition times into and/or out of one or more phases of a seizure may also be determined.

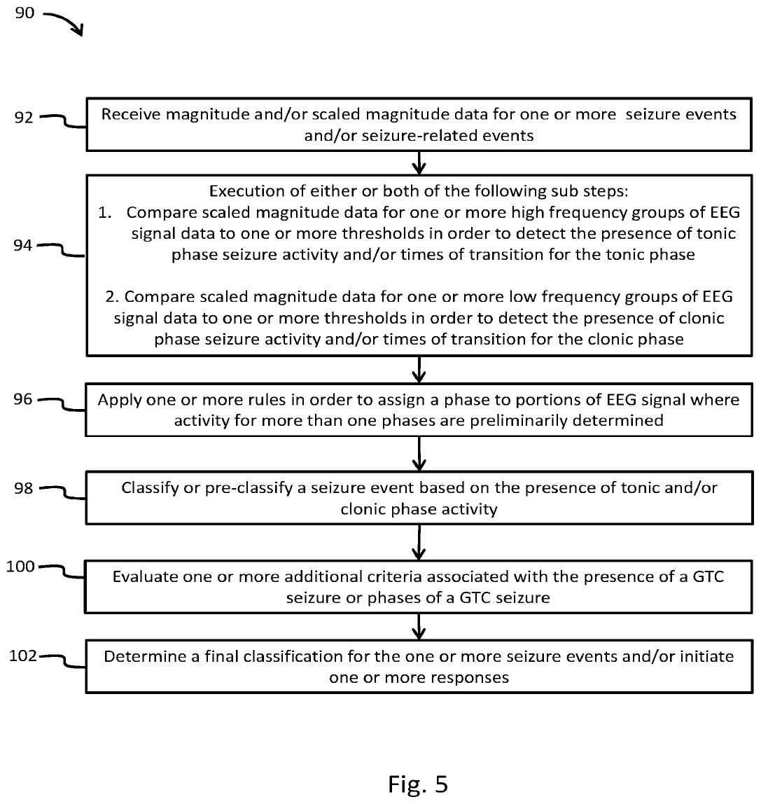

For example, in some embodiments, step 24 and/or step 26 of method 10 may include execution of the sub-routine described in method 90 (shown in FIG. 5). Accordingly, in some embodiments, responses initiated as part of step 102 of the method 90 may be executed as part of one or more responses that may be initiated in step 26 of the method 10. In the step 92, magnitude and/or scaled magnitude data for one or more detected seizure events may be received. For example, where method 90 is executed as a sub-routine in method 10, magnitude and/or scaled magnitude data may be determined as described above, (e.g., the data may include data for one or more high frequency groups of EEG signal and one or more low frequency groups of EEG signal). In some embodiments, seizure events may include one or more seizure events detected, for example, as described above, including in relation to step 14 of method 10.

In some embodiments, as shown in step 94 (see sub-step 1), a scaled magnitude for one or more high frequency groups of EEG signal may be compared to a threshold of about 0.30 to 0.95 in order to identify the tonic phase of a seizure. For example, in some embodiments, if a scaled magnitude of about 0.80 is determined for one or more groups of EEG signal including a high frequency component of an EMG signal, a tonic phase may be recognized. In some embodiments, other thresholds within the above range may be used. For example, and without limitation, in some embodiments, within the above range of thresholds, thresholds of about 0.40, about 0.50, about 0.65, about 0.70, about 0.75, about 0.80, about 0.85, and about 0.90 may be applied.

In some embodiments, a transition time for the start of the tonic phase of a seizure may be identified based on detection of when a scaled magnitude for one or more high frequency groups of EEG signal exceeds a threshold. For example, a transition time may be identified the first time a threshold is met, identified based on when some number of consecutive points meet the threshold, or identified based on some other suitable analysis of data points that may exceed a threshold. In some embodiments, transition out of a tonic phase may include determining when a scaled magnitude for one or more groups of EEG signal data including a high frequency component of an EEG signal fails to exceed a threshold. Alternatively, where a clonic phase follows the tonic phase, the duration of the tonic phase may be based on a determined time of transition into the clonic phase of a seizure, as described below.

In some embodiments, as shown in step 94 (see sub-step 2), a scaled magnitude for one or more low frequency groups of EEG signal may be compared to a threshold of about 0.30 to 0.95 in order to identify the clonic phase of a seizure. For example, in some embodiments, if a scaled magnitude of about 0.80 is determined for one or more groups of EEG signal data including a low frequency component of an EEG signal, a clonic phase may be recognized. In some embodiments, other thresholds within the above range may be used. For example, and without limitation, in some embodiments, within the above range of thresholds, thresholds of about of about 0.40, about 0.50, about 0.65, about 0.70, about 0.75, about 0.80, about 0.85, and about 0.90 may be used.

In some embodiments, a transition time for the start of the clonic phase of a seizure may be identified based on detection of when a scaled magnitude for one or more low frequency groups of EEG signal exceeds a threshold. For example, a transition time may be identified the first time a threshold is met, identified based on when some number of consecutive points meet the threshold, or identified based on some other suitable analysis of data points that may exceed a threshold. In some embodiments, transition out of a clonic phase may include determining when a scaled or unscaled magnitude for one or more groups of EEG signal data including a low frequency component of an EEG signal fails to exceed a threshold.

For some seizure events, during a certain time, such as transition between the phases, activity for each of the tonic and clonic phases of a seizure may be found to exceed one or more of the aforementioned thresholds. Accordingly, more than one phase of a seizure may be preliminarily identified. In some embodiments, as shown in step 96, methods herein may include determining if more than one phase are detected, and if more than one phase are detected, one or more rules may be applied in order to assign a phase. For example, in some embodiments, if both phases are preliminarily found to be active, the phase assigned may be based on one or more ratios between a group of EEG signal including a high frequency component and a group of EEG signal including a low frequency component. For example, in some embodiments, if both phases are found to be active, the phase may be described as tonic, unless the scaled strength of a group of EEG signal data including a low frequency component is found to be greater than about 1.25 times higher than the scaled strength of a group of EMG signal data including a high frequency component. For example, under that scenario, the seizure phase may then be classified as clonic. Alternatively, in some embodiments, no attempt may be made to classify times where both phases of activity are identified.

As shown in step 98, in some embodiments, a seizure event may be classified based on the presence of tonic and/or clonic phase activity. In some embodiments, if both clonic phase activity and tonic phase activity are detected, a seizure may be classified as a GTC seizure. In some embodiments, a seizure-related event may be classified as one including a clonic phase. For example, even if tonic phase activity fails to be detected, one or more responses may be initiated. For example, in some embodiments where the methods 10, 90 are used for real-time detection of seizure activity, one or more emergency or other alarms may be initiated based on detection of clonic phase activity. In some embodiments, a seizure-related event may be classified as one including a tonic phase. For example, even if clonic phase activity fails to be detected, one or more responses may be initiated. For example, at least for some patients or some patients under some conditions, exclusive detection of tonic phase activity may be used to identify seizure events that may not demand an emergency response.

In some embodiments, one or more additional routines may be executed in order to verify a classification made in step 98. Accordingly, a seizure event classified as above may, for example, be referred to as either a classified GTC seizure or pre-classified GTC seizure, based on whether additional classification steps are performed. In some embodiments, final classification of a seizure event that has been pre-classified as a GTC seizure (or other seizure type) may include analysis of one or more additional criteria. For example, in some embodiments, a first group of additional criteria, a second group of additional criteria, a third group of additional criteria, or any combination of the aforementioned additional criteria may be used to classify or verify the classification of a seizure event.

For example, as shown in step 100, in some embodiments, a first group of additional criteria may include whether magnitude data of one or more groups of EEG signal meets one or more thresholds. For example, positive identification of a GTC seizure or a tonic-only event may include verification that magnitude data for one or more groups of EEG signal including a high frequency component of an EEG signal meets or exceeds one or more magnitude thresholds. In some embodiments, the aforementioned magnitude data may be determined from data collected during times preliminarily classified to be part of a tonic phase portion of a seizure. For example, magnitude data may be selected from data included within what was preliminarily identified to be the tonic phase of a seizure. For example, a preliminary identification of the phase may be determined based on a comparison of scaled magnitude data from a high frequency group of EEG signal to one or more thresholds as described above.

Similarly, a positive identification of a GTC seizure or a clonic-only event may include verification that magnitude data for one or more groups of EEG signal data including a high frequency component of an EEG signal meets one or more magnitude thresholds. In some embodiments, the aforementioned magnitude data may be determined from data collected during times preliminarily classified to be part of a clonic phase portion of a seizure. For example, magnitude data may be selected from data included within what was preliminarily identified to be the clonic phase of a seizure. For example, a preliminary identification of the phase may be determined based on a comparison of scaled magnitude data from a low frequency group of EEG signal data to one or more thresholds as described above.

In some embodiments, a first group of additional criteria may be deemed met if a magnitude value threshold is reached for data collected during what was preliminarily classified to be the tonic phase, if a magnitude value threshold is reached for data collected during what was preliminarily classified to be the clonic phase, or if both of the aforementioned conditions are met.

In some embodiments, a second group of additional criteria for classification of a GTC seizure (or other seizure-related event) may include whether one or more times for individual phase duration or total seizure duration meet one or more duration thresholds. For example, a second additional criterion for positive identification of a GTC seizure may include comparison of duration times for the tonic phase of a seizure, the clonic phase of a seizure, the entire seizure, or combinations thereof to one or more duration thresholds. For example, as described above, transition times into and out of one or more phases of a GTC seizure may be determined. Accordingly, duration times for phases of a GTC seizure may be readily determined by calculating the duration between appropriate transition times. And, in some embodiments, a second group of additional criteria for positive identification of a GTC seizure may include comparison of one or more duration times to one or more duration time thresholds (e.g., maximum duration time threshold, minimum duration time threshold, or both).

In some embodiments, a third additional criterion for positive identification of a GTC seizure may include determining whether a ratio between a magnitude of signals included in a high frequency group of EEG signal and a magnitude of signals included in a low frequency group of EEG signal meets one or more ratio thresholds. For example, in some embodiments, an integrated value for the area under a high frequency group of EEG signal may be determined over the course of a seizure event or seizure event pre-classified to be a GTC seizure. For example, a seizure event may be expected to be a GTC seizure because it meets various criteria, including, for example, one or more of the criteria described above. Similarly, an integrated value for the area under a low frequency group of EEG signal data may be determined. Temporal boundaries for integration may be established from one or more transition times into and out of seizure phases (e.g., as may be determined based on comparison of scaled magnitude data to one or more thresholds). Alternatively, integration boundaries with respect to time may be selected in some other convenient way. For example, integration boundaries with respect to time may include some portion of EEG signal selected in step 14, such as all selected data. In some embodiments, a ratio between magnitudes of high and low frequency groups of EEG signal data may be referred to as a qualified area under the curve ratio or QUAC ratio, which may be expressed as shown in Equation 6.

$$QUAC Ratio = \frac{{\int }_{0}^{T} H F ( t ) dt}{{\int }_{0}^{T} LF ( t ) dt} \qquad Equation 6$$

In some embodiments, a QUAC ratio may be determined. If the QUAC ratio is greater than a lower QUAC ratio threshold, verification of the presence of a GTC seizure-related event may be confirmed. For example, a third additional criterion may be deemed satisfied. In some embodiments, a lower QUAC threshold ratio may be about 0.02 to about 0.04. In some embodiments, if the QUAC ratio is within each of a lower QUAC ratio threshold and a higher QUAC ratio threshold, verification of the presence of a GTC seizure-related event may be confirmed. For example, a third additional criterion may be deemed satisfied. In some embodiments, an upper QUAC ratio threshold may be about 0.5 to about 1.0. Of course, other suitable ratios may be defined in order to classify events. For example, in some embodiments, the denominator and numerator of the above QUAC ratio may be interchanged. Similarly, other appropriate ratio thresholds may be used.

As shown in the step 102, a final classification may be determined for any of the one or more seizure-related events analyzed. In some embodiments, the final classification may be the classification made in step 98. For example, no additional group of additional criteria may be evaluated. Alternatively, final classification may include evaluating whether one or more of the additional criteria described in relation to step 100 confirms or contradicts the presence of a pre-classified seizure-related event. For example, in some cases a pre-classified GTC seizure event may be deemed to be of an undetermined seizure-related event type if it fails to meet one or more of the additional group of criteria.

Further in step 102, one or more responses may be initiated. In some embodiments, a response may include organization of classification data and/or other characteristics data for a seizure-related event (e.g., duration times for detected phases) and providing the data to caregivers. For example, one or more reports may be generated.

In some embodiments, methods herein may include detection of when a patient may be experiencing a medical condition that resembles epilepsy, but where the patient may in fact be prone to experience PNES events. For example, in some embodiments useful for diagnosis or verification of a diagnosis that a patient may be suffering from PNES, designated EEG seizure data may be processed in order to classify designated EMG seizure events as either GTC seizures or PNES seizures. In some embodiments, EEG signal data including, for example, raw EEG signal data or sorted EEG signal data, may be analyzed. For example, EEG signal data including seizure-related events may be selected and classified in order to detect PNES seizures.

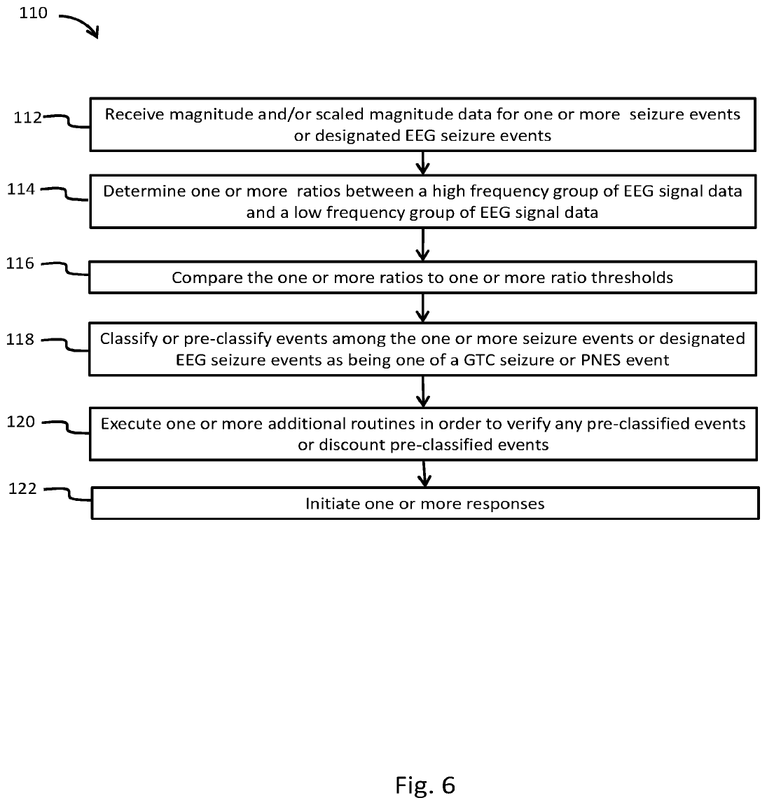

For example, in some embodiments, EEG signal may be evaluated using the method 10, wherein steps included in the sub-routine described in method 110 (shown in FIG. 6) may be included or used in execution of step 24 and/or step 26. For example, as shown in step 112, magnitude and/or scaled magnitude data for one or more detected seizure events or designated EEG seizure events may be received. Thus, in some embodiments, received magnitude and/or scaled magnitude data (step 112) may be derived from designated EEG seizure data. In other embodiments, received magnitude and/or scaled magnitude data (step 112) may be derived from EEG signal data that was selected in step 14 based on one or more seizure-detection routines. For example, in some embodiments, the one or more seizure-detection routines may be configured in order to achieve high selectivity for seizure events. For example, in some embodiments, the one or more seizure-detection routines may be configured in order to maintain high selectivity for detecting seizure events, even if such configuration may be achieved at the expense of detection sensitivity. For example, in some embodiments, the one or more seizure-detection routines applied in step 14 may be based on detection and qualification of samples of EEG signal including elevations, wherein thresholds for seizure detection are suitable for maintaining high selectivity. In some embodiments, received magnitude and/or scaled magnitude data (step 112) may be derived from sorted EEG signal data. For example, as described herein, sorted EEG signal data may be marked by one or more of a caregiver, patient, other persons, and combinations thereof in order to identify seizure events.

In some embodiments, as described in the step 114, one or more ratios between a high frequency group of EEG signal and a low frequency group of EEG signal may be determined. For example, one or more QUAC ratios may be calculated as shown in Equation 6. And, as shown in the step 116, the one or more QUAC ratios may be compared to one or more QUAC ratio thresholds. For example, it may be determined if a QUAC or other suitable ratio (such as an inverted ratio as described below) meets one or more threshold ratio conditions. In some embodiments, as described in step 118, one or more seizure-related events or designated EEG seizure events may be classified based, for example, on the comparison of QUAC ratios and thresholds. For example, in some embodiments, a QUAC ratio may be compared to an upper QUAC ratio threshold of about 0.02 to about 0.04. If the QUAC ratio is less than an upper QUAC ratio threshold, an event (e.g., seizure event or designated seizure event) may be classified as a PNES seizure. In some embodiments, a QUAC ratio may be compared to one or more lower QUAC ratios, and if, for example, the QUAC ratio is greater than the lower QUAC ratio, an event may be classified as a GTC seizure. Other suitable ratios may be defined in order to classify events as GTC and/or PNES events. For example, in some embodiments, the denominator and numerator of the QUAC ratio shown in Equation 6 may be inverted. Accordingly, other appropriate ratio thresholds may be used. For example, in some embodiments wherein the terms in the ratio in Equation 6 are switched, it may be suitable to determine if an inverted QUAC ratio is more than a lower ratio threshold in order to classify a seizure event as a PNES seizure.

Additionally, in some embodiments, as shown in step 120, one or more additional procedures may be initiated to verify a classification that an event should properly be characterized as determined based on the above comparison of one or more QUAC ratios to one or more thresholds. In some embodiments, where one or more additional procedures may be executed to verify a suspected classification, that classification may be referred to as pre-classification.

For example, in some embodiments, in the step 120, one or more steps in method 90 may be executed in order to increase confidence that a seizure classified as a GTC seizure according to method 110 is proper. Alternatively, if the additional procedures do not indicate that the pre-classification was correct, the pre-classification may be discounted or changed. In some embodiments, in the step 120, one or more steps may be executed to verify and/or discount a classification of one or more pre-classified events as PNES events. For example, in some embodiments, one or more routines may be executed to examine whether data suspected as being related to a PNES event is artificially periodic. For example, one or more of the routines further described, for example, in Applicant's U.S. Pat. No. 8,983,591 and associated with periodicity of signal data may be used to verify or discount one or more pre-classified events as being PNES events.

As shown in step 122, one or more responses may be initiated. For example, in some embodiments, classification data may be included in one or more reports which may be provided to a physician or other caregiver.

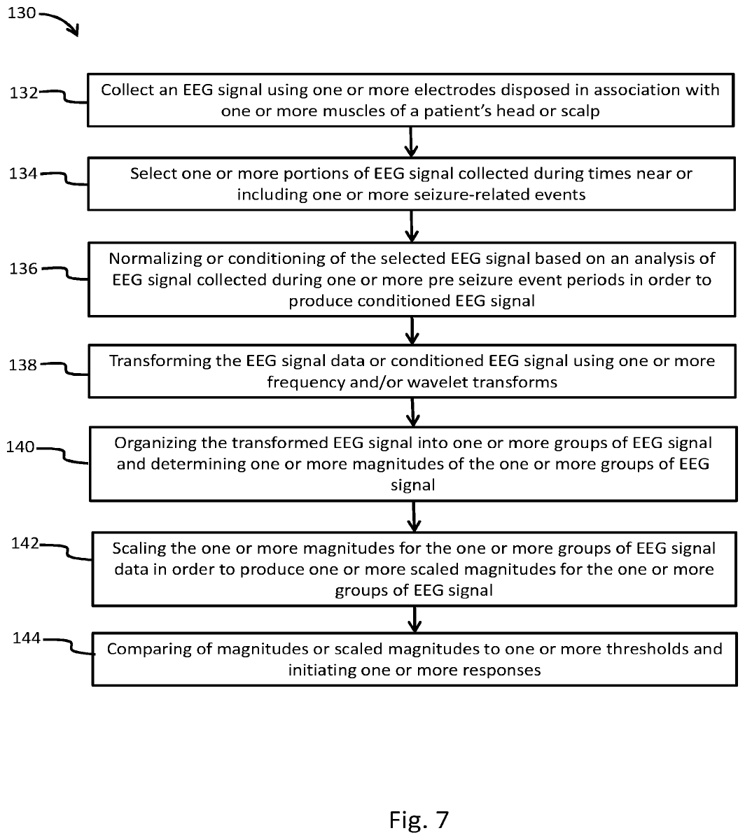

FIG. 7 illustrates some embodiments of a method 130 for analysis of patient medical data collected using one or more sensors, including, for example, sensors which may comprise or consist of EEG sensors. In some embodiments, method 130 may include analysis of collected medical data in real-time and may, for example, be used to initiate an alarm or other response suitable for a detected seizure, type of seizure, or seizure possessing certain characteristics.

As shown in step 132, collection of an EEG signal may include disposing one or more electrodes in association with one or more muscles on the head or scalp of a patient. In some embodiments, the EEG electrodes may be specifically designed to be used in an ambulatory setting.

In some embodiments, collected EEG signal may be processed to provide EEG signal in a form suitable for input and/or processing in a computer processor. For example, in some embodiments, a collected EEG signal may be amplified and processed using an analog-to-digital converter in order to produce digital EEG signal data. In some embodiments, operations such as rectification, low pass filtering, and/or other operations that may be used to shape or condition an EEG signal may also be executed in the step 132.

In some embodiments, as shown in step 134, one or more portions of EEG signal may be selected for further processing. For example, one or more seizure-detection routines may be used to detect one or more seizure-related events, and EEG signals near or including detected seizure-related events may be selected for further processing. In some embodiments, any suitable seizure-detection routine used for selection of EEG signal as described in step 14 of the method 10 may be used in the step 134. In some embodiments, selection of EEG signal in step 134 may include detecting a seizure event indicating the presence or increased risk of a seizure using one or more processors included in a detection device disposed on or near one or more of a patient's muscles. For example, one or more seizure events may be detected. In some embodiments, a detection device may be a device that is minimally intrusive to the patient and which may be configured to allow the patient to freely move during daily activity. In some embodiments, selected EEG signal may be isolated from other EEG signal and sent to a remote processor, such as may be included in a stationary base station, where the selected EEG signal may be further processed in additional steps of method 130. However, in some embodiments, selected data in step 134 may be further processed within the same mobile detection device as used in selection of data in step 134.

In some embodiments, EEG signal selected in step 134 may include data collected during, before, or after a detected seizure event. For example, as further described in step 138, selected data may be further processed using one or more frequency and/or wavelet transforms. In some embodiments, EEG signal selected in step 134 and processing in step 138 may include a predetermined amount of EEG signal data collected adjacent or near a detected seizure-related event. For example, in some embodiments, all data collected over a 5-minute period (or some other suitable predetermined period) may be selected. Alternatively, EEG signal data selected for processing may include all data collected after detection of a seizure-related event in step 134. Or, all EEG signal data collected after detection of a seizure-related event but prior to a stop signal may be selected. For example, if EEG signal data collected after a detected seizure-related event returns to a baseline amplitude level, selection of data for processing may be stopped.

In some embodiments, step 134 may include execution of one or more seizure-detection routines that may run continuously or nearly continuously without drawing large amounts of energy from a battery or other source of energy. For example, as further described in U.S. Provisional Application 62/485,268, which is commonly owned by Applicant, seizure-detection routines that process relatively short segments of an EEG signal (e.g., less than about several seconds of data) in order to determine an amplitude value or some statistical values calculated therefrom, such as a T-squared statistical value or principal component value, may generally operate using limited computational resources and without drawing large amounts of energy from a battery or other source of energy, advantages which may be particularly beneficial when used with patient-worn or personal mobile detection devices where battery and computational or processing resources may be limited.

In some embodiments, a seizure-detection routine may compare one or more property values of an EEG signal to a threshold. For example, some seizure-detection routines may examine one or more short sections of an EEG signal data for the presence of an elevated EEG signal amplitude. If one or more elevated values of EEG signal amplitude are detected that are above one or more thresholds, a response may be almost immediately initiated. In some embodiments, seizure-detection routines executed in the step 134 may evaluate one or more segments of EEG signal data in order to determine an amplitude value or some statistical values calculated therefrom, such as a T-squared statistical value or principal component value. The aforementioned property values may be compared to one or more thresholds in order to determine if a seizure-related event is detected and to select EEG signal data in the step 134.

In some embodiments, one or more seizure-detection routines may be used to detect a time or time range for the start of seizure activity. Once a start time or time range for the start of a seizure is determined, a portion of data that includes the start of the seizure and/or a portion of data that includes one or more pre-seizure time periods may be selected. For example, if detection of a seizure-related event identifies that a seizure may have occurred sometime within about the last 60 seconds before an estimated start time of the seizure-related event (or other range consistent with the temporal resolution for detection of a seizure-related event), a pre-seizure-related event may be selected from data collected about 60 seconds or more before that estimated start time of the seizure-related event. Accordingly, one or more pre-seizure periods may be divided out and used in further processing. For example, statistical informational calculated from one or more pre-seizure periods may be used to normalize or condition EEG signal data, as described in the step 136.

In some embodiments, as shown in step 136, selected data may be normalized or conditioned. For example, normalization or conditioning of data may include steps as described in step 16 of method 10.

As shown in step 138, EEG signal data or conditioned EEG signal data may be processed using one or more frequency and/or wavelet transforms. In some embodiments, the one or more frequency and/or wavelet transforms may execute over some predetermined interval or until some stop signal is triggered to prevent further selection of data for processing. In some embodiments, a wavelet transform used in step 138 may be one suitable for application in real-time detection. For example, a Morlet Wavelet or other suitable wavelet may be used.

As shown in step 140, EEG signal may be organized in one or more groups, and magnitudes of signals in the one or more groups may be determined. For example, in some embodiments, data may be organized in one or more collections of bins. The collections may, for example, include one or more high frequency collections of bins and one or more low frequency collections of bins as described in detail in step 20 of the method 10.

As shown in step 142, magnitude data may be scaled. Scaling of magnitude data may include dividing magnitude data for a group of EEG signal data by a maximum magnitude value achieved for the group of EEG signal data over some time period. As further described in step 22 of the method 10, scaling of magnitude data may include determining one or more absolute maximum magnitude values or one or more local maximum magnitude values. For example, when scaling magnitude data collected over time, one or more local maximum magnitude values may be determined and used to scale data. And, in some embodiments, methods herein may determine if a local maximum magnitude value meets requirements to be designated as an absolute maximum magnitude value. For example, in some embodiments, if a local maximum magnitude is maintained for greater than about 5 seconds to about 10 seconds (e.g., no other adjacent or following values exceed the local maximum magnitude value), then the local maximum magnitude may be designated as an absolute maximum magnitude value. In some embodiments, other information, such as the slope or shape of EEG signal data on either side of a local/absolute maximum magnitude value may also be used in determining if a magnitude value is designated as a local or absolute magnitude value. In some embodiments, one or more responses initiated in step 144 may be made only if scaled magnitude values have been determined to be based on an absolute maximum magnitude value.

As shown in step 144, magnitudes or scaled magnitudes for one or more groups of EMG signals may be compared to one or more thresholds. Based on the comparison, one or more responses may be initiated.

In the method 130, determined magnitudes and scaled magnitude of EEG signal data (steps 140, 142) may be determined across time. For example, organization of EEG signal data in one or more groups of EEG signal data (step 140), calculation of magnitude data for the organized groups (step 140), and scaling of magnitude data (step 142) may be performed over one or more time intervals. The process may then be repeated for other intervals of time during an analysis. Continuously or periodically within this process, such as every 1 second or at other suitable time interval, magnitude and/or scaled magnitude data may be analyzed and one or more responses may be initiated. For example, in some embodiments, one or more alarms may be initiated if a seizure-related event is determined to be a GTC or other seizure event type based on one or more of the characterization steps described herein.

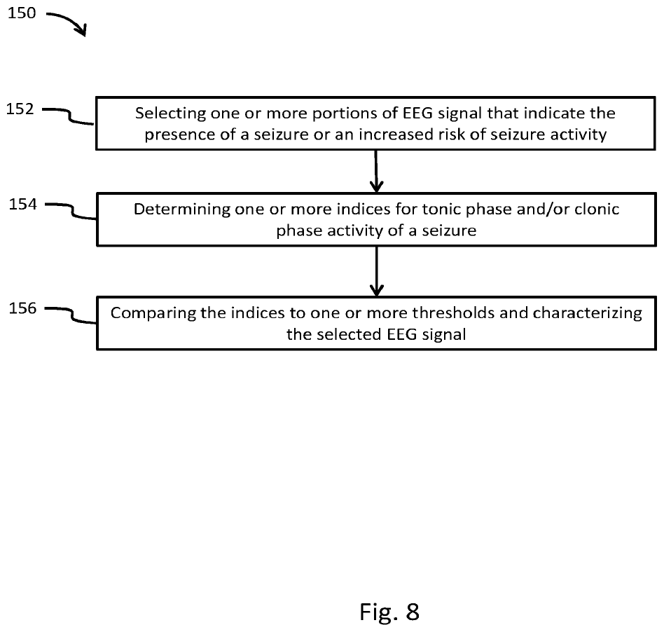

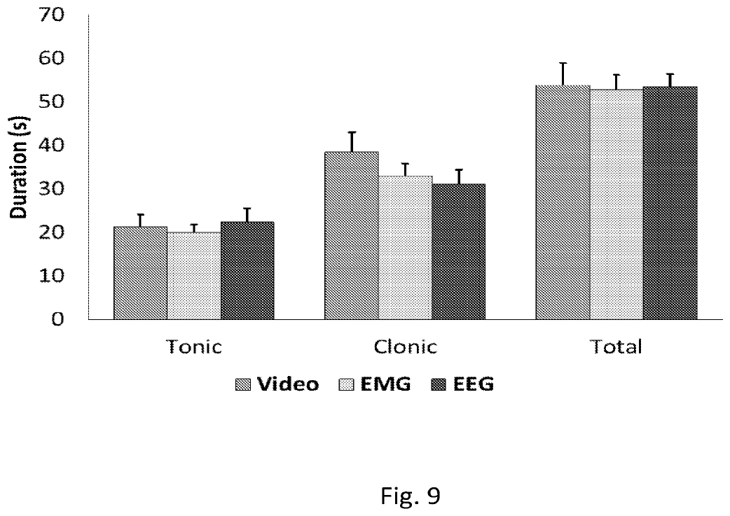

FIG. 8 illustrates embodiments of a method 150 for processing EEG signal. Method 150 may, for example, be run independently of method 10 or used in combination with the method 10. For example, indices for the tonic and clonic phases as described herein may be used help characterize whether a GTC seizure or other activity is present. The method 150 may be used to distinguish some detected events that may be indicative of non-seizure sources from true seizures. For example, some non-seizure events may be detected by some seizure-detection routines and may be characterized by the presence of high magnitude data that may be large but not sustained over time. Method 150 may be used to identify such activity and used to discriminate non-seizure events from true GTC seizures.

In a step 152, method 150 may include selection of one or more portions of EEG signal. Selection of one or more portions of EEG signal data may include detecting, at least at some level of probability, one or more seizure events or one or more events indicating increased risk of seizure occurrence. Selection of data that may include a seizure is further described in greater detail in, for example, step 14 of the method 10. For example, in some embodiments, an about 10-minute portion of data may be selected and used for processing in the step 152.

In a step 154, one or more indices of tonic phase and/or clonic phase activity of a seizure may be calculated. For example, indices of seizure activity for the tonic and clonic phases of a seizure may be calculated as shown in Equation 7 and in Equation 8, respectively.

I_{T}=k1∫(Magnitude(High Freq.)dt where, 0<t<x Equation 7

I_{C}=k2∫(Magnitude(Low Freq.)dt where, 0<t<x Equation 8

In Equation 7, the tonic phase index (I_{T}) includes a scaling factor k1 and an integrated value across time for signal magnitude calculated for a high frequency group of EEG signal. For example, magnitude data as described in step 20 of method 10 may be included in Equation 7. In other embodiments, scaled magnitude data (as may be determined in the step 22 of method 10) may be used. In Equation 8, the clonic phase index (I_{C}) includes a scaling factor k2 and an integrated value across time for signal magnitude calculated for a lower frequency group of EEG signal. In other embodiments, scaled magnitude data (as may be determined in the step 22 of method 10) may be used. Operations and steps associated with determining of magnitude and/or scaled magnitude data (as used in Equations 7 and 8), such as, for example, wavelet processing and normalization, are described in greater detail in reference to method 10.

In step 156, indices for the tonic and clonic phases may be used to characterize the selected EEG signal data. For example, in some embodiments, for a dataset to be characterized as a true GTC seizure, both the tonic and clonic indices may exceed a threshold value. In some embodiments, scaling factors k1 and k2 may be selected so that a threshold value of 1 may be used to indicate characterization of tonic and/or clonic phase activity. In some embodiments, indices for the tonic and clonic phases of seizures may be evaluated across time or during one or more analysis windows. For example, in Equations 7 and 8, magnitude values for the high and low frequency groups of EEG signal data may be evaluated over an interval of about 10 minutes. However, in some embodiments, indices for tonic and clonic activity may be evaluated continuously over time or at some number of discrete times. Accordingly, characterization of signals (step 156) may also be executed across time. For example, in some embodiments, indices may be evaluated at regular intervals following times identified when a seizure or possible seizure may have occurred. For example, at regular intervals following detection of seizure or possible seizure activity, such as at intervals of between about 30 seconds to about 240 seconds, indices for tonic and clonic phase activity may be evaluated. In some of those embodiments, scaling factors k1 and k2 may depend upon time (e.g., the scaling factors k1 and k2 may change during the progression of a suspected seizure) and may be described as k1(t) and k2(t).

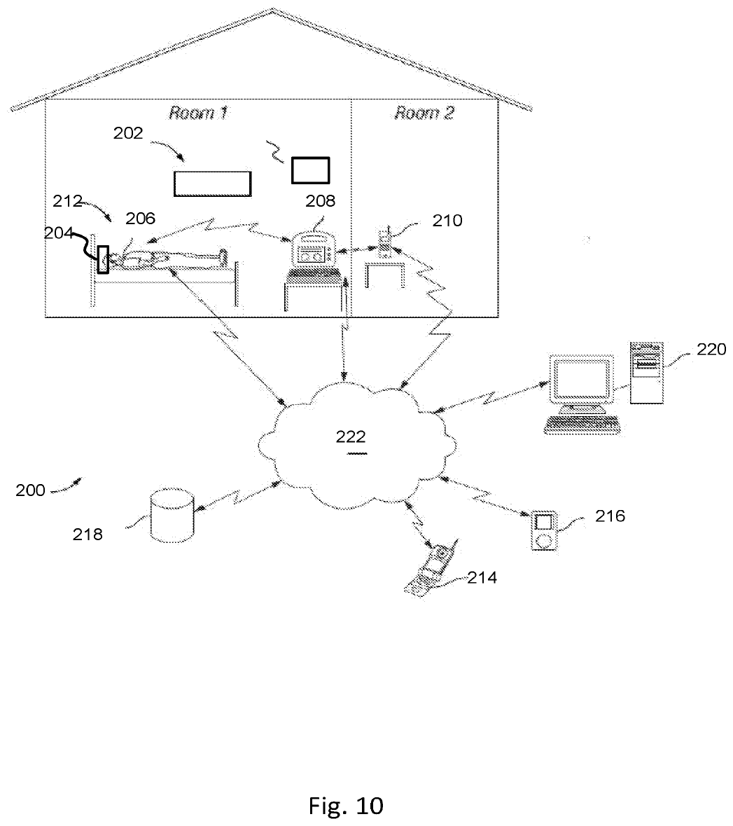

A variety of suitable systems may be used for collecting EEG and other patient-related data, organizing such data for system optimization, and analyzing seizure event data. FIG. 10 illustrates an exemplary embodiment of such a system (which may, for example, include detection capability for an EMG signal) that may be configured to monitor a patient for seizure activity using the methods described herein.

In the embodiment of FIG. 10, a seizure detection or analysis system 200 may include a video camera 202, an EEG detection unit 204, an EMG detection unit 206, a base station 208, and an alert transceiver 210. The EEG detection unit 204 may comprise one or more EEG electrodes capable of detecting physiological signals and delivering those signals to one or more processors for processing. The EMG detection unit 206 may comprise one or more EMG electrodes capable of detecting electrical signals from muscles at or near the skin surface of a patient 212 and delivering those electrical EMG signals to a processor for processing. The EMG electrodes may be attached to the patient 212, and may, in some embodiments, be implanted within the tissue of the patient 212 near a muscle that may be activated during a seizure. Implanted devices may, for example, be particularly amenable for some patients where EMG signals may typically be weak, such as patients with significant adipose tissue. The base station 208 may comprise a computer capable of receiving and processing EEG signals, EMG signals, or both from the detection units 204, 206, and determining from the processed EEG and/or EMG and EEG signals whether a seizure may have occurred, and sending an alert to a caregiver. The alert transceiver 210 may be carried by, or placed near, a caregiver to receive and relay alerts transmitted by the base station 208 or transmitted directly from one or more of the detection units 204, 206. Other components that may be included in the system 200, including for example, wireless communication devices 214 and 216, storage database 218, and remote computer 220. The devices may be connected, including, for example, over wireless network 222.

In some embodiments, systems herein may be configured differently. For example, a system may include an EEG detection unit configured to collect EEG signals and one or more processors configured to receive the signals and analyze the signals using the methods described herein. In some embodiments, systems herein may include one or more processors configured to receive and analyze EEG signals and optionally one or more storage databases including EEG data.

Additional details of some suitable systems which may, for example, be used for collecting large amounts of patient-related data, organizing such data for system optimization or for execution of database queries, and initiating an alarm or other response based on suspected seizure activity are described in the references incorporated herein. For example, Applicant's U.S. Pat. No. 8,983,591 (incorporated herein by reference) includes a detailed description of apparatus components which may be used in some of the embodiments herein when coupled with an EEG detection unit and including processors designed to process EEG signals.

Additional information related to the methods and apparatus described herein may be understood in connection with the examples provided below.

### EXAMPLE 1

In this Example 1, a study was performed to test whether EEG signals may be used to characterize seizure activity based on an algorithm developed for analysis of muscle activity. In the study, a total of 196 patients were monitored at 11 different epilepsy monitoring facilities. EEG signals were collected together with EMG recordings (collected at patient's biceps). A total of 29 GTC seizures were recorded (as determined by two out of three ABPN certified epileptologists), with two separate types of amplifiers, including a mixture of primary and secondary GTC seizures. The data further shown herein was calculated with a group of 15 detected GTC seizures, which includes all detected seizures when using a first type of amplifier in this study.

EMG data was collected using a GTC seizure monitoring and alarming system collecting data at a frequency of 1 kHz. The EMG data was processed in real-time by an algorithm previously validated. EEG electrodes were placed using the international 10-20 system. Among the EEG electrodes, data was selected from the F7, F8, T3, and T4 electrodes, which are the electrodes placed near the frontalis and temporalis muscles. Notably, signals from some of the other EEG electrodes, more distant from the frontalis and temporalis muscles, did not produce useful muscle activity data. The EEG signals were recorded at sampling rates from 200-1024 Hz. The temporal location of the GTC seizures in the recording and the duration of both the bilateral appendicular tonic and clonic phase activity were annotated based on both the video and the EEG recording by three neurologists board certified by the ABPN in Epilepsy Sub-speciality.

The single channel EMG signal from the biceps and the four channels of the EEG signal were each processed and used to determine various characteristics of seizure activity. For example, the duration of the clonic phase of recorded seizures was determined using a continuous wavelet transform applied using the Morlet wavelet basis function using coefficients which represent lower frequency activity (2-58.5 Hz). Coefficients from each transformed EEG signal were summated at each point in time. Following wavelet transformation, magnitude data was compared to threshold in order to determine transition points into and out of the tonic phase. A total duration of each GTC seizure was also determined using an envelope filter created to measure the dynamic root mean square (RMS) of the signal (e.g., the signal including data up to about 200 Hz). In this Example 1, tonic phase durations were calculated by subtracting the duration of the clonic phase from the total seizure duration. The duration of the GTC seizure, the duration of the clonic phase, and the duration of the tonic phase calculated using EMG electrodes and each of the above-mentioned EEG electrodes were then compared using the paired student t-test with Bonferroni-Holm correction for multiple statistical comparisons.

Table 1, shown below, shows a general breakdown of data collected in this Example 1.

TABLE 1

| Breakdown of | | | |
|---|---|---|---|
| Data used for | | | |
| Analysis | Total GTCS | Primary GTCS | Secondary GTCS |
| GTCS Detected | 29 | 4 | 25 |
| by Epileptologists & | | | |
| Device | | | |
| Total Events used | 15* | 2 | 13 |
| for Wavelet and | | | |
| RMS Analyses | | | |

*EEG was recorded using Wave Amplifier (Natus Medical Inc., Pleasanton, California)

A statistical comparison was made between GTCS total duration, clonic phase duration, and tonic phase duration as calculated by (1) expert review of video review (2) expert review of EMG and (3) EEG wavelet analysis as described. Using paired t-test statistical analysis with bonferoni-holm correction, it was found that the video, EMG, and EEG seizure semiological analyses were not significantly different (Table 2, P>0.05). Table 2, shown below, includes a quantitative data for this analysis.

TABLE 2

| | | | | Clonic phase | | | | | |
|---|---|---|---|---|---|---|---|---|---|
| | Total phase duration | | | duration | | | Total GTCS duration | | |
| | Video | EMG | EEG | Video | EMG | EEG | Video | EMG | EEG |
| Mean | 21.27 | 19.91 | 22.36 | 38.46 | 33.00 | 31.09 | 53.91 | 52.91 | 53.46 |
| (s) | | | | | | | | | |
| Standard | 2.85 | 1.91 | 3.23 | 4.48 | 2.75 | 3.31 | 4.98 | 3.36 | 2.95 |
| Error of | | | | | | | | | |
| the Mean | | | | | | | | | |

Although the methods and apparatuses disclosed and their advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition, or matter, means, methods and steps described in the specification. Use of the word “include,” for example, should be interpreted as the word “comprising” would be, i.e., as open-ended. As one will readily appreciate from the disclosure, processes, machines, manufactures, compositions of matter, means, methods, or steps presently existing or later to be developed that perform substantially the same function or achieve substantially the same result as the corresponding embodiments described herein may be utilized. Accordingly, the appended claims are intended to include within their scope such processes, machines, manufactures, compositions of matter, means, methods or steps.

We claim:

1. A method of analyzing an EEG signal for characteristics of seizure activity comprising:
receiving an EEG signal for analysis;
wherein said EEG signal is selected or filtered in order to increase a prevalence of a part of said EEG signal derived from activation of muscle activity;
wherein said EEG signal includes at least one seizure event;
transforming one or parts of said EEG signal using one or more frequency or wavelet transforms in order to produce transformed data;
determining one or more magnitudes or scaled magnitudes for one or more frequency bands included among said transformed data;
analyzing said one or more magnitudes or scaled magnitudes in order to identify one or more phases of at least one seizure event among said at least one seizure event.

2. The method of claim 1 wherein said EEG signal is provided from EEG data stored in one or more databases.

3. The method of claim 1 further comprising executing one or more seizure-detection routines in order to detect said at least one seizure event.

4. The method of claim 1 wherein said at least one seizure event includes a designated seizure event previously identified by one or more caregivers as being a seizure event.

5. The method of claim 1 wherein said EEG signal is a signal collected from one or more EEG electrodes positioned at one or more of the F7, F8, T3, and T4 positions.

6. The method of claim 1 wherein said EEG signal is a signal collected from one or more EEG electrodes positioned over or near one of the frontalis muscles, temporalis muscles, or both.

7. The method of claim 1 wherein said one or more frequency bands includes a first group of one or more frequency bands including a frequency range from about 2 Hz to about 70 Hz and a second group of frequency bands including a frequency range above about 100 Hz.

8. The method of claim 1 wherein said one or more frequency bands includes a frequency band including a frequency range above about 100 Hz; and further comprising:
comparing a scaled magnitude determined for said frequency band to a threshold in order to determine one or more transition times into or out of a tonic phase of a seizure.

9. The method of claim 1 wherein said one or more frequency bands includes a frequency band including a frequency range from about 2 Hz to about 70 Hz, and further comprising:
comparing a scaled magnitude determined for said frequency band to a threshold in order to determine one or more transition times into or out of a clonic phase of a seizure.

10. The method of claim 1 further comprising calculating one or more qualified areas under a curve based on said scaled magnitudes; and
identifying if at least one seizure event among said at least one seizure event is either of a generalized-tonic-clonic seizure or a psychogenic nonepileptic seizure.

11. A system for analyzing an EEG signal for characteristics of seizure activity comprising:
one or more processors configured to:
receive an EEG signal;
wherein said EEG signal is selected or filtered in order to increase a prevalence of a part of said EEG signal derived from activation of muscle activity;
wherein said EEG signal includes at least one seizure event;
transform one or parts of said EEG signal using one or more frequency or wavelet transforms in order to produce transformed data;
determine one or more magnitudes or scaled magnitudes for one or more frequency bands included among said transformed data;
analyze said one or more magnitudes or scaled magnitudes in order to identify one or more phases of at least one seizure event among said at least one seizure event.

12. The system of claim 11 wherein said one or more frequency bands includes a first group of one or more frequency bands including a frequency range from about 2 Hz to about 70 Hz and a second group of frequency bands including a frequency range above about 100 Hz.

13. The system of claim 11 wherein said one or more frequency bands includes a frequency band including a frequency range above about 100 Hz, and wherein said processor is further configured to:
compare a scaled magnitude determined for said frequency band to a threshold in order to determine one or more transition times into or out of a tonic phase of a seizure.

14. The system of claim 11 wherein said one or more frequency bands includes a frequency band including a frequency range from about 2 Hz to about 70 Hz, and further comprising:
comparing a scaled magnitude determined for said frequency band to a threshold in order to determine one or more transition times into or out of a clonic phase of a seizure.

15. The system of claim 11 wherein said processor is further configured to calculate one or more qualified areas under a curve based on the scaled magnitudes; and
identify if at least one seizure event among said at least one seizure event is either of a generalized-tonic-clonic seizure or a psychogenic nonepileptic seizure.
